(19) United States
(12) Patent Application Publication
(10) Pub. No.: US 2022/0192899 A1
(43) Pub. Date: Jun. 23, 2022

(54) ABSORBENT ARTICLE

(71) Applicant: KIMBERLY-CLARK WORLDWIDE, INC., Neenah, WI (US)

(72) Inventors: WunDuk Lee, Appleton, WI (US); Vinod Chaudhary, Alpharetta, GA (US); Benjamin C. Orkiszewski, Alpharettta, GA (US); Ryan E. Bowers, Appleton, WI (US); Aster Kammrath, Neenah, WI (US)

(21) Appl. No.: 17/606,801

(22) PCT Filed: May 7, 2019
(86) PCT No.: PCT/US2019/031033

§ 371 Date: Oct. 27, 2021

(51) Int. Cl. A61F 13/53; B01J 20/26; B01J 20/28

(52) U.S. Cl. CPC A61F 13/53; B01J 20/267; B01J 20/28016; B01J 20/28004; A61F 2013/530788; B01J 2220/68; A61F 2013/530569; A61F 2013/5307; A61F 2013/530737; B01J 2220/445

(57) ABSTRACT

The present invention provides for an absorbent article that includes a topsheet, backsheet and absorbent core. The absorbent core includes a fibrous material and a particulate superabsorbent polymer composition. The superabsorbent polymer composition exhibits advantageous performance of absorption rate, surface tension, bulk density, centrifuge retention capacity, absorbency under load, gel bed permeability and particle size.

### BACKGROUND OF THE DISCLOSURE

A super absorbent polymer (SAP) is a synthetic polymer material capable of absorbing moisture from about 500 to about 1,000 times its own weight, and each manufacturer has denominated it as different names such as SAM (Super Absorbency Material), AGM (Absorbent Gel Material) or the like. Such super absorbent polymers started to be practically applied in sanitary products, and now they are widely used for production of personal care absorbent articles such as disposable diapers for infants and children, training pants, youth pants, feminine hygiene products and adult incontinence garments or the like.

For personal care absorbent articles, the super absorbent polymer is generally mixed with a fluff/pulp material to form absorbent cores. In recent years, however, efforts have been made to provide personal care absorbent articles having a thinner thickness. As a part of such efforts, the development of so-called pulpless diapers and the like in which the content of pulp is reduced or pulp is not used at all is being actively advanced.

As described above, in the case of personal care absorbent articles in which the pulp content is reduced or the pulp is not used, a super absorbent polymer is contained at a relatively high ratio and these super absorbent polymer particles are inevitably contained in one or multiple layers in the absorbent articles. In order for the super absorbent polymer particles contained in the one or multiple layers to absorb liquid such as urine more efficiently, the super absorbent polymer needs to basically exhibit faster absorption rate with high absorption capacity and liquid permeability.

Hence, in recent years, attempts have been continuously made to prepare and provide a super absorbent polymer exhibiting an improved absorption rate.

The most common method for increasing the absorption rate may be a method of widening the surface area of the super absorbent polymer by either forming a porous structure inside the super absorbent polymer and/or reducing size of super absorbent polymer particles.

In order to widen the surface area of the super absorbent polymer in this way, conventionally, a method of forming a porous structure in a base polymer powder by performing the crosslinking polymerization using a carbonate foaming agent, or a method of forming the porous structure by introducing bubbles into a monomer mixture in the presence of a surfactant and/or a dispersing agent and then performing crosslinking polymerization, and the like, have been applied. In addition, it has been tried to reduce size of super absorbent polymer particles to widen surface area.

However, it was difficult to achieve the absorption rate of a certain level or higher with keeping high absorption capacity and liquid permeability, which is critical for the absorbencies of personal care absorbent articles, by any method previously known in the art.

Furthermore, conventional methods inevitably involve the use of an excessive amount of foaming agents and/or surfactants in order to obtain a super absorbent polymer having more improved absorption rate. As a result, they showed disadvantages of various physical properties such as surface tension, particle size, liquid permeability or bulk density of the super absorbent polymer being greatly reduced.

Thus, there remains a continuing need to provide faster absorption rate with high absorption capacity and liquid permeability for a super absorbent polymer composition that will result in an advantageous performance when the super absorbent polymer is incorporated into an absorbent article.

### SUMMARY OF THE DISCLOSURE

The present invention provides for an absorbent article that includes a topsheet, backsheet and absorbent core. The absorbent core has both a fibrous material and a particulate superabsorbent polymer composition. The particulate superabsorbent polymer composition exhibits advantageous performance at a defined absorption rate, surface tension, bulk density, centrifuge retention capacity, absorbency under load, gel bed permeability and particle size.

In one embodiment, the present invention is directed to an absorbent article that includes a topsheet, backsheet and an absorbent core disposed between the topsheet and backsheet. The absorbent core includes a fibrous material and a particulate superabsorbent polymer composition. The particulate superabsorbent polymer composition includes a base polymer powder including a first cross-linked polymer of a water-soluble ethylenically unsaturated monomer having an acidic group of superabsorbent polymer composition. The particulate superabsorbent polymer composition has an absorption rate (also known as “vortex time”) measured by a Vortex Time test method of 5 to 35 seconds, a surface tension of 65 to 72 mN/m, and a bulk density of 0.50 to 0.65 g/ml, a centrifuge retention capacity (CRC) of 23 g/g or more, an absorbency under load (AUL) at 0.9 psi of 14 g/g or more, a gel bed permeability (GBP) of 10 darcies or more and a particle size of 150 to 850 μm. Further, articles of the particulate superabsorbent polymer composition having a particle size of 600 μm or more make up less than 12% by weight of the composition and particles having a particle size of 300 μm or less make up less than 20% by weight of the composition.

### BRIEF DESCRIPTION OF DRAWINGS

FIG. 1 illustrates a partially cut away, top plan view of an absorbent article in a stretched and laid flat condition with the surface of the article that contacts the skin of the wearer facing the viewer.

### DEFINITIONS

When introducing elements of the present disclosure or the preferred embodiment(s) thereof, the articles “a”, “an”, and “the” are intended to mean that there are one or more of the elements.

The terms “comprising”, “including” and “having” are intended to be inclusive and mean that there may be additional elements other than the listed elements.

The term “absorbent article” refers to devices that absorb and contain body exudates, and, more specifically, refers to devices that are placed against or in proximity to the body of the wearer to absorb and contain the various exudates discharged from the body. Absorbent articles may include diapers, pant diapers, open diapers, diaper covers having fastening means for fastening the diaper, training pants, adult incontinence undergarments, feminine hygiene products, breast pads, care mats, bibs, wound dressing products, and the like. As used herein, the term “body exudates” includes, but is not limited to, urine, blood, vaginal discharges, breast milk, sweat and fecal matter.

The term “absorbent core” for the purposes of the present invention is preferably understood as meaning a construction which in the case of an absorbent article, for instance a diaper, may be arranged between the upper ply, impermeable to aqueous fluids and facing away from the body side of the wearer, and the lower ply, permeable to aqueous fluids and facing the body side of the wearer, and the primary function of which is to absorb and store the fluids, for example blood or urine, which have been imbibed by the absorbent article. The absorbent core itself preferably comprises no imbibition system, no upper ply and no lower ply of the absorbent article.

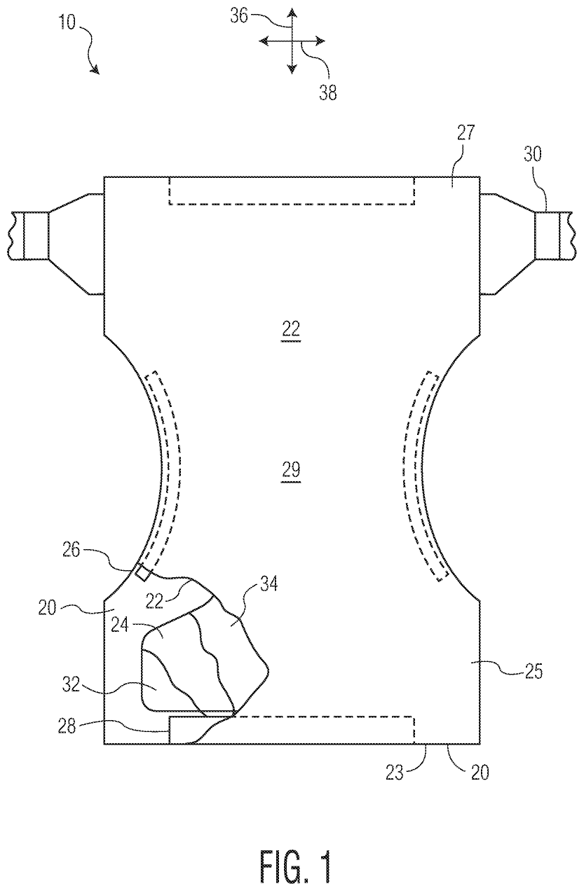

The term “longitudinal” and “transverse” have their customary meaning, as indicated by the longitudinal and transverse axes depicted in FIG. 1. The longitudinal axis lies in the plane of the article and is generally parallel to a vertical plane that bisects a standing wearer into left and right body halves when the article is worn. The transverse axis lies in the plane of the article generally perpendicular to the longitudinal axis.

The term “polymer” includes, but is not limited to, homopolymers, copolymers, for example, block, graft, random, and alternating copolymers, terpolymers, etc., and blends and modifications thereof. Furthermore, unless otherwise specifically limited, the term “polymer” shall include all possible configurational isomers of the material. These configurations include, but are not limited to isotactic, syndiotactic, and atactic symmetries.

The term “superabsorbent polymer” as used herein refers to water-swellable, water-insoluble organic or inorganic materials including superabsorbent polymers and superabsorbent polymer compositions capable, under the most favorable conditions, of absorbing at least about 10 times their weight, or at least about 15 times their weight, or at least about 25 times their weight in an aqueous solution containing 0.9 weight percent sodium chloride.

The term “superabsorbent polymer composition” as used herein refers to a superabsorbent polymer comprising a surface crosslinking agent in accordance with the present invention.

The term “surface crosslinking” as used herein refers to the level of functional crosslinks in the vicinity of the surface of the superabsorbent polymer particle, which is generally higher than the level of functional crosslinks in the interior of the superabsorbent polymer particle. As used herein, “surface” describes the outer-facing boundaries of the particle.

The terms “particle,” “particulate,” and the like, when used with the term “superabsorbent polymer composition,” refer to the form of discrete units. The units may comprise flakes, fibers, agglomerates, granules, powders, spheres, pulverized materials, or the like, as well as combinations thereof. The particles can have any desired shape: for example, cubic, rod like polyhedral, spherical or semi-spherical, rounded or semi-rounded, angular, irregular, et cetera.

### DETAILED DESCRIPTION

The current disclosure relates to an absorbent article having a topsheet, a backsheet and an absorbent core disposed between the topsheet and backsheet. The absorbent core contains particulate superabsorbent polymer compositions which absorb water, aqueous liquids, blood and the like. The particulate superabsorbent polymer compositions of the invention have superior performance properties and will be described in further detail herein. First, a description of a typical absorbent article with which the particulate superabsorbent polymer compositions may be used is provided.

A typical absorbent article will be explained with reference to FIG. 1. FIG. 1 illustrates an exemplary disposable absorbent article 10 that is an infant disposable diaper employing the particulate superabsorbent polymer composition of the invention. The example of the use of the particulate superabsorbent polymer composition in a disposable diaper for infants is intended to be representative and not limiting; the particulate superabsorbent polymer compositions of the invention may be used similarly with other types and constructions of absorbent articles. The disposable absorbent article 10 includes a backsheet or (outer cover) 20, a liquid permeable topsheet (or bodyside liner) 22 positioned in facing relation with the backsheet 20, and an absorbent core 24, such as an absorbent pad, that is located between the topsheet 22 and the backsheet 20. The article 10 has an outer surface 23, a front waist region 25, a back waist region 27, and a crotch region 29 connecting the front and back waist regions 25, 27. The backsheet 20 defines a length and a width that, in the illustrated aspect, coincide with the length and width of the article 10. The absorbent core 24 generally defines a length and width that are less than the length and width of the backsheet 20, respectively. Thus, marginal portions of the article 10, such as marginal sections of the backsheet 20, can extend past the terminal edges of the absorbent core 24. In the illustrated aspects, for example, the backsheet 20 extends outwardly beyond the terminal marginal edges of the absorbent core 24 to form side margins and end margins of the article 10. The topsheet 22 is generally coextensive with the backsheet 20 but can optionally cover an area that is larger or smaller than the area of the backsheet 20, as desired. In other words, the topsheet 22 is connected in superposed relation to the backsheet 20. The backsheet 20 and topsheet 22 are intended to face the garment and body of the wearer, respectively, while in use.

To provide improved fit and to help reduce leakage of body exudates from the article 10, the article side margins and end margins can be elasticized with suitable elastic members, such as single or multiple strands of elastic. The elastic strands can be composed of natural or synthetic rubber and can optionally be heat shrinkable or heat elasticizable. For example, as representatively illustrated in FIG. 1, the article 10 can include leg elastics 26 that are constructed to operably gather and shirr the side margins of the article 10 to provide elasticized leg bands that can closely fit around the legs of the wearer to reduce leakage and provide improved comfort and appearance. Similarly, waist elastics 28 can be employed to elasticize the end margins of the article 10 to provide elasticized waists. The waist elastics 28 are configured to operably gather and shirr the waist sections to provide a resilient comfortably close fit around the waist of the wearer. In the illustrated aspects, the elastic members are illustrated in their uncontracted, stretched condition for the purpose of clarity.

Fastening means, such as hook and loop fasteners 30, may be employed to secure the article 10 on a wearer. Alternatively, other fastening means, such as buttons, pins, snaps, adhesive tape fasteners, cohesives, mushroom-and-loop fasteners, a belt, and so forth, as well as combinations including at least one of the foregoing fasteners can be employed. Additionally, more than two fasteners can be provided, particularly if the article 10 is to be provided in a prefastened configuration.

The article 10 may further include other layers between the absorbent core 24 and the topsheet 22 or backsheet 20. For example, article 10 may also include a surge management layer 34 located between the topsheet 22 and the absorbent core 24 to prevent pooling of the fluid exudates and further improve air exchange and distribution of the fluid exudates within the article 10.

The article 10 may be of various suitable shapes. For example, the article 10 may have an overall rectangular shape, T-shape or an approximately hourglass shape. In the shown aspect, the article 10 has a generally I-shape. The article 10 further defines a longitudinal direction 36 and a transverse direction 38. Other suitable article components that can be incorporated on absorbent articles include containment flaps, waist flaps, elastomeric side panels, and the like. Examples of possible article configurations are described in U.S. Pat. No. 4,798,603 issued Jan. 17, 1989, to Meyer et al.; U.S. Pat. No. 5,176,668 issued Jan. 5, 1993, to Bernardin; U.S. Pat. No. 5,192,606 issued Mar. 9, 1993, to Proxmire et al., and U.S. Pat. No. 5,509,915 issued Apr. 23, 1996 to Hanson et al.

The various components of the article 10 are integrally assembled employing various types of attachment mechanisms such as adhesive, sonic bonds, thermal bonds, and so forth, as well as combinations including at least one of foregoing mechanisms. In the shown aspect, for example, the topsheet 22 and backsheet 20 are assembled to the absorbent core 24 with lines of adhesive, such as a hot melt, pressure-sensitive adhesive. Similarly, other article components, such as the elastic members 26 and 28, fastening members 30, and surge layers 34 can be assembled into the article 10 by employing the above-identified attachment mechanisms.

The backsheet 20 of the article 10 may include any material used for such applications, such as a substantially vapor-permeable material. The permeability of the backsheet 20 may be configured to enhance the breathability of the article 10 and to reduce the hydration of the wearer's skin during use without allowing excessive condensation of vapor, such as urine, on the garment facing surface of the backsheet 20 that can undesirably dampen the wearer's clothes. The backsheet 20 can be constructed to be permeable to at least water vapor and can have a water vapor transmission rate of greater than or equal to about 1,000 grams per square meter per 24 hours (g/m²/24 hr). For example, the backsheet 20 can define a water vapor transmission rate of about 1,000 to about 6,000 g/m²/24 hr.

The backsheet 20 is also desirably substantially liquid impermeable. For example, the backsheet 20 can be constructed to provide a hydrohead value of greater than or equal to about 60 centimeters (cm), or, more specifically, greater than or equal to about 80 cm, and even more specifically, greater than or equal to about 100 cm. A suitable technique for determining the resistance of a material to liquid penetration is Federal Test Method Standard (FTMS) 191 Method 5514, dated Dec. 31, 1968.

As stated above, the backsheet 20 may include any material used for such applications, and desirably includes materials that either directly provide the above desired levels of liquid impermeability and air permeability and/or materials that can be modified or treated in some manner to provide such levels. The backsheet 20 can be a nonwoven fibrous web constructed to provide the required level of liquid impermeability. For example, a nonwoven web including spunbond and/or meltblown polymer fibers can be selectively treated with a water repellent coating and/or laminated with a liquid impermeable, vapor permeable polymer film to provide the backsheet 20. In another aspect, the backsheet 20 can include a nonwoven web including a plurality of randomly deposited hydrophobic thermoplastic meltblown fibers that are sufficiently bonded or otherwise connected to one another to provide a substantially vapor permeable and substantially liquid impermeable web. The backsheet 20 can also include a vapor permeable nonwoven layer that has been partially coated or otherwise configured to provide liquid impermeability in selected areas. In yet another example, the backsheet 20 is provided by an extensible material. Further, the backsheet 20 material can have stretch in the longitudinal 36 and/or transverse 38 directions. When the backsheet 20 is made from extensible or stretchable materials, the article 10 provides additional benefits to the wearer including improved fit.

The topsheet 22, employed to help isolate the wearer's skin from liquids held in the absorbent core 24, can define a compliant, soft, non-irritating feel to the wearer's skin. Further, the topsheet 22 can be less hydrophilic than the absorbent core 24, to present a relatively dry surface to the wearer, and can be sufficiently porous to be liquid permeable, permitting liquid to readily penetrate through its thickness. A suitable topsheet 22 may be manufactured from a wide selection of web materials, such as porous foams, reticulated foams, apertured plastic films, natural fibers (for example, wood or cotton fibers), synthetic fibers (for example, polyester or polypropylene fibers), and the like, as well as a combination of materials including at least one of the foregoing materials.

Various woven and nonwoven fabrics may be used for the topsheet 22. For example, the topsheet 22 may include a meltblown or spunbond web (e.g., of polyolefin fibers), a bonded-carded web (e.g., of natural and/or synthetic fibers), a substantially hydrophobic material (e.g., treated with a surfactant or otherwise processed to impart a desired level of wettability and hydrophilicity), and the like, as well as combinations including at least one of the foregoing. For example, the topsheet 22 can include a nonwoven, spunbond, polypropylene fabric, optionally including about 2.8 to about 3.2 denier fibers formed into a web having a basis weight of about 22 grams per square meter (g/m²) and a density of about 0.06 gram per cubic centimeter (g/cc).

The absorbent core 24 of the article 10 may include a matrix of hydrophilic fibers, such as a fibrous web of cellulosic fibers, mixed with particles of the particulate superabsorbent polymer composition. The wood pulp fluff can be exchanged with synthetic, polymeric, meltblown fibers, and the like, as well as a combination including at least one of the foregoing. The particulate superabsorbent polymer composition can be substantially homogeneously mixed with the hydrophilic fibers or can be nonuniformly mixed. Alternatively, the absorbent core 24 can include a laminate of fibrous webs and particulate superabsorbent polymer composition and/or a suitable matrix for maintaining the particulate superabsorbent polymer composition in a localized area. When the absorbent core 24 includes a combination of hydrophilic fibers and the particulate superabsorbent polymer, the hydrophilic fibers and particulate superabsorbent polymer composition can form an average basis weight for the absorbent core 24 that may be about 300 grams per square meter (g/m²) to about 900 g/m², or, more specifically, about 500 g/m² to about 800 g/m², and even more specifically, about 550 g/m² to about 750 g/m².

In general, the particulate superabsorbent polymer composition is present in the absorbent core 24 in an amount of greater than or equal to about 50 weight percent (wt percent), or, more desirably greater than or equal to about 70 wt percent, based on a total weight of the absorbent core 24. For example, in a particular aspect, the absorbent core 24 can include a laminate that includes greater than or equal to about 50 wt percent, or, more desirably, greater than or equal to about 70 wt percent of particulate superabsorbent polymer composition overwrapped by a fibrous web or other suitable material for maintaining the high-absorbency material in a localized area.

Optionally, the absorbent core 24 may further include a support (e.g., a substantially hydrophilic tissue or nonwoven wrap sheet (not illustrated)) to help maintain the integrity of the structure of the absorbent core 24. The tissue wrapsheet may be placed about the web/sheet of high-absorbency material and/or fibers, optionally over at least one or both major facing surfaces thereof. The tissue wrapsheet can include an absorbent cellulosic material, such as creped wadding or a high wet-strength tissue. The tissue wrapsheet may optionally be configured to provide a wicking layer that helps to rapidly distribute liquid over the mass of absorbent fibers constituting the absorbent core 24. If this support is employed, the colorant 40 may optionally be disposed in the support, on the side of the absorbent core 24 opposite the backsheet 20.

Due to the thinness of absorbent core 24 and the high absorbency material within the absorbent core 24, the liquid uptake rates of the absorbent core 24, by itself, can be too low, or cannot be adequately sustained over multiple insults of liquid into the absorbent core 24. To improve the overall liquid uptake and air exchange, the article 10 can further include a porous, liquid-permeable layer or surge management layer 34, as representatively illustrated in FIG. 1. The surge management layer 34 is typically less hydrophilic than the absorbent core 24, and can have an operable level of density and basis weight to quickly collect and temporarily hold liquid surges, to transport the liquid from its initial entrance point and to substantially completely release the liquid to other parts of the absorbent core 24. This configuration can help prevent the liquid from pooling and collecting on the portion of the article 10 positioned against the wearer's skin, thereby reducing the feeling of wetness by the wearer. The structure of the surge management layer 34 can also enhance the air exchange within the article 10.

Various woven and nonwoven fabrics may be used to construct the surge management layer 34. For example, the surge management layer 34 can be a layer including a meltblown or spunbond web of synthetic fibers (such as polyolefin fibers); a bonded-carded-web or an airlaid web including, for example, natural and/or synthetic fibers; hydrophobic material that is optionally treated with a surfactant or otherwise processed to impart a desired level of wettability and hydrophilicity; and the like, as well as combinations including at least one of the foregoing. The bonded carded-web can, for example, be a thermally bonded web that is bonded using low melt binder fibers, powder, and/or adhesive. The layer can optionally include a mixture of different fibers. For example, the surge management layer 34 can include a hydrophobic, nonwoven material having a basis weight of about 30 to about 120 g/m².

The backsheet 20 desirably comprises a material that is substantially liquid impermeable, and may be elastic, stretchable or nonstretchable. The backsheet 20 may be a single layer of liquid impermeable material, but desirably comprises a multi-layered laminate structure in which at least one of the layers is liquid impermeable. For instance, the backsheet 20 may include a liquid permeable outer layer and a liquid impermeable inner layer that are suitably joined together by a laminate adhesive (not shown). Suitable laminate adhesives, which may be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J., U.S.A. The liquid permeable outer layer can be any suitable material and desirably one that provides a generally cloth-like texture. One example of such a material is a 20 gsm (grams per square meter) spunbond polypropylene nonwoven web. The outer layer may also be made of those materials of which liquid permeable topsheet 22 is made. While it is not a necessity for outer layer to be liquid permeable, it is desired that it provides a relatively cloth-like texture to the wearer.

The inner layer of the backsheet 20 may be both liquid and vapor impermeable, or may be liquid impermeable and vapor permeable. The inner layer is desirably manufactured from a thin plastic film, although other flexible liquid impermeable materials may also be used. The inner layer, or the liquid impermeable backsheet 20 when a single layer, prevents waste material from wetting articles, such as bedsheets and clothing, as well as the wearer and caregiver. A suitable liquid impermeable film for use as a liquid impermeable inner layer, or a single layer liquid impermeable backsheet 20, is a 1.0 mil polyethylene film commercially available from Edison Plastics Company of South Plainfield, N.J., U.S.A. If the backsheet 20 is a single layer of material, it can be embossed and/or matte finished to provide a more cloth-like appearance. As earlier mentioned, the liquid impermeable material can permit vapors to escape from the interior of the disposable absorbent article, while still preventing liquids from passing through the backsheet 20. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn., U.S.A.

The liquid permeable topsheet 22 is illustrated as overlying the backsheet 20 and may but need not have the same dimensions as the backsheet 20. The topsheet 22 is desirably compliant, soft feeling, and non-irritating to the child's skin.

The topsheet 22 may be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics may be used for the topsheet 22. For example, the topsheet may be composed of a meltblown or spunbonded web of polyolefin fibers. The topsheet may also be a bonded-carded web composed of natural and/or synthetic fibers.

The topsheet 22 may be composed of a substantially hydrophobic material, and the hydrophobic material may, optionally, be treated with a surfactant or otherwise processed to impart a desired level of wettability and hydrophilicity. For example, the material may be surface treated with about 0.28 weight percent of a surfactant commercially available from the Rohm and Haas Co. under the trade designation Triton X-102. The surfactant may be applied by any conventional means, such as spraying, printing, brush coating or the like. The surfactant may be applied to the entire topsheet 22 or can be selectively applied to particular sections of the topsheet 22, such as the medial section along the longitudinal centerline.

Alternatively, a suitable liquid permeable topsheet 22 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber available from CHISSO Corporation, Osaka, Japan. In this particular bicomponent fiber, the polypropylene forms the core and the polyethylene forms the sheath of the fiber. Other fiber orientations are possible, such as multi-lobe, side-by-side, end-to-end, or the like. While the backsheet 20 and topsheet 22 may comprise elastomeric materials, it can be desirable in some embodiments for the composite structure to be generally inelastic, where the top sheet, the backsheet 20 and the absorbent core 24 comprise materials that are generally not elastomeric.

Suitable elastic materials are described in the following U.S. patents: U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. In particular embodiments, the elastic material comprises a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Mormon; and European Patent Application No. EP 0 217 032 published on Apr. 8, 1987 in the names of Taylor et al.; all of which are incorporated herein by reference.

The absorbent core 24 may include suitable superabsorbent polymers (or materials) capable of absorbing moisture may be selected from natural, synthetic, and modified natural polymers and materials. The superabsorbent materials can be inorganic materials, such as silica gels, or organic compounds, such as crosslinked polymers. The absorbent articles 10 of the invention include a particulate superabsorbent polymer composite with unique performance properties that will be described herein. The particulate superabsorbent polymer composition may be used alone or in combination with other absorbent materials in the absorbent core 24. For example, the particulate superabsorbent polymer composite may be used in combination with one or more of standard superabsorbent polymers and pulp fiber.

The particulate super absorbent polymer composition of the invention may be manufactured by either of two processes by itself or in combination of those processes.

One of the processes identified hereinafter as “Process A”, for sake of clarity, is for preparing a super absorbent polymer composition that includes the steps of

1) preparing a monomer mixture including a water-soluble ethylenically unsaturated monomer having an acidic group of which at least a part is neutralized, anionic surfactant having an HLB value of 20 to 40 at a concentration of 50 to 200 ppmw, an internal crosslinking agent, and a polymerization initiator, wherein the monomer mixture is formed by a method comprising a step of mixing a solution containing the anionic surfactant with a mixture containing the monomer and the internal crosslinking agent while passing the solution through a tubular flow channel having a plurality of projecting pins therein at a space velocity of 50 to 1500 1500 (min⁻¹),

2) performing crosslinking polymerization to form hydrogel polymer,

3) drying, pulverizing and classifying the hydrogel polymer to form a base polymer powder, and

4) further crosslinking the surface of the base polymer powder in the presence of a surface crosslinking agent to form a surface cross-linked layer.

In the preparation of Process A, anionic surfactants satisfying specific HLB values are included in the monomer mixture, wherein the monomer mixture is formed by mixing an anionic surfactant solution with a mixture containing the monomer and the internal crosslinking agent while passing the solution through a particular type of tubular flow channel at a space velocity of 50 to 1500 (min⁻¹).

When a solution containing an anionic surfactant is mixed with a monomer or the like to form a monomer mixture in this way, formation of bubbles in the solution can be greatly promoted while the solution containing the anionic surfactant collides with a plurality of protruding pins in the tubular flow channel. Moreover, due to the action of the fixed amount of anionic surfactant, the bubbles can be highly stabilized, and such bubbles can be retained in a large amount within the monomer mixture.

As a result, when crosslinking polymerization is performed using the monomer mixture formed by the method of Process A, it was confirmed that formation of bubbles is promoted compared to any conventional method, and thus a base polymer powder and a super absorbent polymer having a highly developed porous structure can be produced.

Therefore, according to Process A, as it has a highly developed porous structure, a particulate superabsorbent polymer composition exhibiting a further improved absorption rate may be produced. Furthermore, it has been found that since the use of a carbonate-based foaming agent may be omitted and the amount of the anionic surfactant used is also relatively reduced, other physical properties of the particulate superabsorbent polymer composition, such as surface tension, liquid permeability or bulk density may be maintained excellently.

The present invention also provides for a method for preparing a super absorbent polymer composition, identified hereinafter as “Process B” that includes the steps of

1) preparing a monomer composition which includes a water-soluble ethylenically unsaturated monomer having an acidic group of which at least a part is neutralized, an internal crosslinking agent and a polymerization initiator,

2) generating bubbles in aqueous solutions using a microbubble generator, and introducing inorganic fine particles into the aqueous solution with bubbles, followed by generating microbubbles by using ultrasonication

3) mixing the aqueous solution in which the microbubbles have been generated and the monomer composition, followed by crosslinking polymerization to form a hydrogel polymer

4) drying, pulverizing and classifying the hydrogel polymer to form a base polymer powder and

5) further crosslinking the surface of the base polymer powder in the presence of a surface crosslinking agent to form a surface crosslinked layer.

Hereinafter, the preparation methods of Processes A and B and the particulate superabsorbent composition obtained therefrom will be described in more detail.

In the preparation of Process A, the water-soluble ethylenically unsaturated monomer may be any monomer commonly used for the preparation of a super absorbent polymer material. As a non-limiting example, the water-soluble ethylenically unsaturated monomer may be a compound represented by the following Chemical Formula 1:

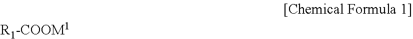

in Chemical Formula 1,

R₁ is an alkyl group having 2 to 5 carbon atoms containing an unsaturated bond,

M¹ is a hydrogen atom, a monovalent or divalent metal, an ammonium group or an organic amine salt.

Preferably, the monomer may be one or more compounds selected from (meth)acrylic acid, and monovalent metal salts, divalent metal salts, ammonium salts, and organic amine salts of these acids. When a (meth)acrylic acid and/or a salt thereof is used as the water-soluble ethylenically unsaturated monomer in this way, it is advantageous in that a super absorbent polymer having improved water absorptivity is obtained. In addition, as the monomer, maleic anhydride, fumaric acid, crotonic acid, itaconic acid, 2-acryloyl ethane sulfonic acid, 2-methacryloyl ethane sulfonic acid, 2-(meth)acryloyl propane sulfonic acid, or 2-(meth)acrylamide-2-methylpropane sulfonic acid, (meth)acrylamide, N-substituted (meth)acrylate, 2-hydroxyethyl(meth)acrylate, 2-hydroxypropyl(meth)acrylate, methoxypolyethyleneglycol(meth)acrylate, polyethyleneglycol(meth)acrylate, (N,N)-dimethylaminoethyl(meth)acrylate, (N,N)-dimethylaminopropyl(meth)acrylamide, and the like may be used.

Here, the water-soluble ethylenically unsaturated monomer may be those having an acidic group of which at least a part is neutralized. Preferably, the monomer may be those in which the monomer is partially neutralized with a basic substance such as sodium hydroxide, potassium hydroxide, ammonium hydroxide or the like.

In this case, the degree of neutralization of the monomer may be 55 to 95 mol %, or 60 to 80 mol %, or 65 to 75 mol %. The range of the degree of neutralization may vary depending on the final physical properties. An excessively high degree of neutralization causes the neutralized monomers to be precipitated, and thus polymerization may not readily occur, whereas an excessively low degree of neutralization not only greatly deteriorates the absorbency of the polymer but also endows the polymer with hard-to-handle properties, like elastic rubber.

For example, the monomer mixture containing the monomer may be provided in a solution state such as an aqueous solution. The concentration of the water-soluble ethylenically unsaturated monomer in the monomer mixture may be properly controlled, in consideration of a polymerization time and reaction conditions, and for example, the concentration may be 20 to 90% by weight, or 40 to 65% by weight.

This concentration range may be advantageous for using gel effect phenomenon occurring in the polymerization reaction of a high-concentration aqueous solution to eliminate a need for removing the unreacted monomer after the polymerization and also for improving pulverization efficiency in pulverization process of the polymer described below. However, if the concentration of the monomer is too low, the yield of the super absorbent polymer may become low. On the contrary, if the concentration of the monomer is too high, there is a process problem that a part of the monomers is precipitated, or pulverization efficiency is lowered upon pulverization of the polymerized hydrogel polymer, and the physical properties of the super absorbent polymer may be reduced.

Meanwhile, the above-mentioned monomers may be mixed together with an anionic surfactant having an HLB value of 20 to 40 and an internal crosslinking agent in a solvent such as an aqueous solvent to form a monomer mixture.

As the anionic surfactant, any ionic surfactant known to have the HLB value may be used. Examples of such anionic surfactants may be one or more selected from sodium dodecyl sulfate, ammonium lauryl sulfate, sodium laureth sulfate, dioctyl sodium sulfosuccinate, perfluorooctane sulfonate, perfluorobutane sulfonate, alkyl-aryl ether phosphate, alkyl ether phosphate, sodium myreth sulfate and carboxylate salt.

Such anionic surfactant may be contained at a concentration of 50 to 200 ppmw, or 60 to 190 ppmw, or 70 to 180 ppmw in the monomer mixture. If the concentration of the anionic surfactant is too low, the absorption rate becomes insufficient, and if the concentration of the anionic surfactant is too high, the other physical properties of the super absorbent polymer such as absorbency under load, liquid permeability, surface tension or bulk density may be deteriorated.

Meanwhile, the monomer mixture may further contain 0.01 wt % or less, or 0% to 0.01 wt %, or 0.001% to 0.007 wt %, of the nonionic surfactant having an HLB value of 4 to 15 in addition to the anionic surfactant. Due to the additional inclusion of such nonionic surfactants, the porous structure of the particulate superabsorbent polymer composition may be further developed, thus further improving its absorption rate.

As the nonionic surfactant, any nonionic surfactant known to have the HLB value may be used. Examples of such nonionic surfactants may be one or more selected from fatty acid ester, sorbitan trioleate, polyethoxylated sorbitan monooleate (product name: TWEEN 80), sorbitan monooleate (product name: SPAN 80) and sugar ester (product name: S-570).

Further, an internal crosslinking agent is further included in the monomer mixture. As the internal crosslinking agent, any compound can be used as long as it enables introduction of a crosslink bond upon polymerization of the water-soluble ethylenically unsaturated monomer. Non-limiting examples of the internal crosslinking agent may include multifunctional crosslinking agents, such as N,N′-methylenebisacrylamide, trimethylolpropane tri(meth)acrylate, ethylene glycol di(meth)acrylate, polyethylene glycol(meth)acrylate, propylene glycol di(meth)acrylate, polypropylene glycol(meth)acrylate, butanediol di(meth)acrylate, butylene glycol di(meth)acrylate, diethylene glycol di(meth)acrylate, hexanediol di(meth)acrylate, triethylene glycol di(meth)acrylate, tripropylene glycol di(meth)acrylate, tetraethylene glycol di(meth)acrylate, dipentaerythritol pentacrylate, glycerin tri(meth)acrylate, pentaerythritol tetraacrylate, triarylamine, ethylene glycol diglycidyl ether, propylene glycol, glycerin, or ethylene carbonate, which may be used alone or in combination of two or more thereof, but are not limited thereto.

Such an internal crosslinking agent may be added at a concentration of about 0.001 to 1% by weight based on the monomer mixture. That is, when the concentration of the internal crosslinking agent is too low, the absorption rate of the composition is lowered and the gel strength may be weakened, which is not preferable. Conversely, when the concentration of the internal crosslinking agent is too high, the absorption capacity of the composition is lowered, which may be undesirable as an absorbent material.

Meanwhile, the monomer mixture, for example, the monomer aqueous solution may further contain one or more additive selected from a polyvalent metal salt, a photoinitiator, a thermal initiator, and a polyalkylene glycol-based polymer, in addition to the above-mentioned monomer, internal crosslinking agent and surfactant.

Such additive may be used to further improve the liquid permeability or the like of the super absorbent polymer (polyvalent metal salt or polyalkylene glycol-based polymer, etc.), or to smooth the crosslinking polymerization and further improve the physical properties of the particulate superabsorbent polymer composition.

The above-mentioned additives may be used in an amount of 2000 ppmw or less, or 0 to 2000 ppmw, or 10 to 1000 ppmw, or 50 to 500 ppmw, based on 100 parts by weight of the monomer, depending on their respective roles. Thereby, it is possible to further improve the physical properties such as the absorption rate, liquid permeability, and absorption performance of the particulate superabsorbent polymer composition.

As the polyalkylene glycol-based polymer among the above-mentioned additives, polyethylene glycol, polypropylene glycol, or the like may be used.

In addition, as the photo (polymerization) initiator and/or the thermal (polymerization) initiator, any polymerization initiator commonly used for the preparation of a superabsorbent polymer may be used. Particularly, even in the case of the photo-polymerization method, a certain amount of heat is generated by ultraviolet irradiation or the like. Further, as the polymerization reaction, which is an exothermic reaction, proceeds, a certain amount of heat is generated and thus, a photo (polymerization) initiator and/or a thermal (polymerization) initiator may be used together to prepare a superabsorbent polymer having more excellent absorption rate and various physical properties.

As the thermal (polymerization) initiator, one or more compounds selected from a persulfate-based initiator, an azo-based initiator, hydrogen peroxide, and ascorbic acid may be used. Specific examples of the persulfate-based initiator may include sodium persulfate (Na₂S₂O), potassium persulfate (K₂S₂O₈), ammonium persulfate (NH₄)₂S₂O₈), and the like. Further, examples of the azo-based initiator may include 2,2-azobis-(2-amidinopropane)dihydrochloride, 2,2-azobis-(N,N-dimethylene)isobutyramidine dihydrochloride, 2-(carbamoylazo)isobutylonitrile, 2,2-azobis[2-(2-imidazolin-2-yl)propane]dihydrochloride, 4,4-azobis-(4-cyanovaleric acid) and the like. More various thermal polymerization initiators are well disclosed in “Principle of Polymerization” written by Odian, (Wiley, 1981), p 203, which may be incorporated herein by reference.

Further, the photo (polymerization) initiator may be, for example, one or more compounds selected from benzoin ether, dialkyl acetophenone, hydroxyl alkylketone, phenyl glyoxylate, benzyl dimethyl ketal, acyl phosphine and α-aminoketone. As the specific example of acyl phosphine, commercially available Lucirin TPO, namely, 2,4,6-trimethyl-benzoyl-trimethyl phosphine oxide, may be used. More various photo-polymerization initiators are well disclosed in “UV Coatings: Basics, Recent Developments and New Applications” written by Reinhold Schwalm, (Elsevier, 2007), p 115, which may be incorporated herein by reference.

Such polymerization initiator may be added at a concentration of 500 ppmw or less, based on 100 parts by weight of the monomer. That is, if the concentration of the polymerization initiator is too low, the polymerization rate becomes low and thus a large amount of residual monomers may be extracted from the final product, which is not preferable. On the contrary, if the concentration of the polymerization initiator is higher than the above range, the polymer chains constituting the network becomes short, and thus the content of water-soluble components is increased and physical properties of the polymer may deteriorate such as a reduction in absorbency under load, which is not preferable.

Meanwhile, in addition to the above-mentioned respective components, the monomer mixture may further contain additives such as a thickener, a plasticizer, a preservation stabilizer, and an antioxidant, if necessary.

The monomer mixture may be prepared in the form of a solution in which the raw materials such as the above-mentioned monomers are dissolved in a solvent. In this case, as the usable solvent, any solvent may be used without limitations in the constitution, as long as it is able to dissolve the above raw materials. Examples of the solvent that can be used include water, ethanol, ethylene glycol, diethylene glycol, triethylene glycol, 1,4-butanediol, propylene glycol, ethylene glycol monobutyl ether, propylene glycol monomethyl ether, propylene glycol monomethyl ether acetate, methyl ethyl ketone, acetone, methyl amyl ketone, cyclohexanone, cyclopentanone, diethylene glycol monomethyl ether, diethylene glycol ethylether, toluene, xylene, butyrolactone, carbitol, methyl cellosolve acetate, N,N-dimethylacetamide, or a mixture thereof.

The above-mentioned monomer mixture having the form of an aqueous solution or the like can be controlled so that the initial temperature has a temperature of 30 to 60° C., and the light energy or thermal energy is applied thereto to perform the crosslinking polymerization.

The monomer mixture, according to process A, may be formed by a method including the steps of forming a primary mixture in a solution state containing the water-soluble ethylenically unsaturated monomer and an internal crosslinking agent; mixing the primary mixture with a basic aqueous solution to form a secondary mixture in which at least a part of the acid groups of the unsaturated monomer is neutralized; and generating a large amount of bubbles while passing a solution containing a nonionic surfactant having an HLB value of 4 to 15, and a solution containing an initiator, other additives and an anionic surfactant through a tubular flow channel having a plurality of projecting pins therein at a space velocity of 50 to 1500 (min⁻¹), or 200 to 1300 (min⁻¹), or 300 to 1000 (min⁻¹), followed by mixing with the secondary mixture containing the neuralized monomer.

In the final stage of such method, more suitably, nonionic surfactants that are not well mixed with other components other than the monomer due to hydrophobicity can be first mixed, and the anionic surfactant for promoting/stabilizing the generation of bubbles in the monomer may be finally added and mixed.

Further, in order to achieve the concentration range of the anionic surfactant in the above-mentioned monomer mixture, in the step of adding and mixing the solution containing the anionic surfactant, it can be proceeded by a method comprising the steps of supplying an aqueous solution containing the anionic surfactant at a concentration of 0.1 to 0.3% by weight, followed by mixing with the secondary mixture containing the neutralized monomer.

Due to Process A forming the monomer mixture, the generation of bubbles in the monomer mixture is further promoted/stabilized, and thus the absorption rate of the super absorbent polymer of the particulate superabsorbent polymer composition may be further improved.

In particular, in the above-mentioned Process A, the generation of bubbles is highly activated while the solution containing the anionic surfactant is passed through a tubular flow channel having a plurality of projecting pins therein at constant space velocity, and such solution can be mixed with other components such as monomers to form a monomer mixture. Therefore, the super absorbent polymer produced by the method of one embodiment can exhibit a greatly improved absorption rate.

In the step of generating a large amount of bubbles by the above-mentioned mixing, it is possible to use a commercialized mixing apparatus having a tubular flow channel having the projecting pins. As an example of such a commercialized mixing apparatus, there can be mentioned a microbubble generator (manufactured by “O2 Bubble”).

Meanwhile, nano-sized microbubbles in the monomer composition can be generated separately in following two steps with addition of inorganic fine particles in the middle of those steps to enhance the stability of bubbles generated, described as preparation of Process B in the present invention. Thereby, even if a surfactant is not contained or it is contained, it is possible to improve the absorption rate while compensating for the drawbacks associated with the use of the surfactant such as reduction of the surface tension, by including only a small amount of 150 ppmw or less.

In addition, the monomer composition according to one embodiment of Process B in the present invention may not contain a forming agent such as sodium bicarbonate which was used to generate bubbles by chemical methods in a conventional method of preparing a super absorbent polymer. In this manner, as the foaming agent is not used, the gel strength of the super absorbent polymer can be kept high.

Meanwhile, in addition to the above-mentioned respective components, the monomer composition may further contain additives such as a thickener, a plasticizer, a preservation stabilizer, and an antioxidant, if necessary.

The monomer composition may be prepared in the form of a solution in which the raw materials such as the above-mentioned monomers are dissolved in a solvent. In this case, as the usable solvent, any solvent may be used without limitations in the constitution, as long as it is able to dissolve the above raw materials. Example of the solvent that can be used include water, ethanol, ethylene glycol, diethylene glycol, triethylene glycol, 1,4-butanediol, propylene glycol, ethylene glycol monobutyl ether, propylene glycol monomethyl ether, propylene glycol monomethyl ether acetate, methyl ethyl ketone, acetone, methyl amyl ketone, cyclohexanone, cyclopentanone, diethylene glycol monomethyl ether, diethylene glycol ethylether, toluene, xylene, butyrolactone, carbitol, methyl cellosolve acetate, N,N-dimethylacetamide, or a mixture thereof.

Next, bubbles are generated in the monomer composition or another aqueous solution (or water) prepared as described above using a microbubble generator.

More specifically, bubbles are first generated in the above-mentioned monomer composition or another aqueous solution or water using a microbubble generator.

In the bubble generating step using the microbubble generator, an available microbubble generator may be a commercialized device without limitation. Preferably, OB-750S, which is a microbubble generator manufactured by O2 Bubble, can be mentioned.

With this microbubble generator, bubbles having a diameter of several microns to several hundred microns are primarily formed in the monomer composition or the aqueous solution. However, there are no or a few amounts of surfactants in the monomer composition or the aqueous solution, bubbles generated in this way do not have enough life-time and thus it is difficult to form a sufficient porous structure.

According to an embodiment of Process B in the present invention, inorganic fine particles are introduced into the aqueous solution in which the bubbles have been generated, and microbubbles are generated using ultrasonication with respect to the monomer composition or aqueous solution into which the inorganic fine particles have been introduced.

By introducing inorganic fine particles into the monomer composition or the aqueous solution in which micro-sized bubbles are generated as described above, and again generating bubbles using ultrasonication, the micro-sized bubbles previously generated are changed to microbubbles having a size of several nanometers to several hundred nanometers, and microbubbles produced due to the inorganic fine particles attached to these bubbles can be maintained in a stable form for a long time.

According to one embodiment of Process B in the present invention, the inorganic fine particles may include one or more selected from the group consisting of silica, clay, alumina, a silica-alumina composite, and titania. These inorganic fine particles may be used in a powdery form or in a liquid form, and in particular, silica powder, alumina powder, silica-alumina powder, titania powder, or a nanosilica solution may be used.

Further, the particle size of the inorganic fine particles is in the range of several tens to several hundred nanometers, which may be about 500 nm or less, or about 300 nm or less, and about 10 nm or more, or about 20 nm or more, or about 40 nm or more. When the particle size of the inorganic fine particles is too small, it causes little generation of bubbles, and when the particle size is too large, formation of bubbles can be rather suppressed.

Further, the inorganic fine particles may be added at a concentration of about 0.05 part by weight or more, or about 0.1 part by weight or more, and about 1 part by weight or less and about 0.5 part by weight or less, based on 100 parts by weight of the water-soluble ethylenic unsaturated monomer. When the amount of the inorganic fine particles used is too small, the absorption rate may be reduced, and when the amount of the inorganic fine particles used is too large, permeability properties may be deteriorated. From such a viewpoint, it may be preferable to use it within the above weight range.

The ultrasonic devices may use commercially available devices without limitation. When using a separate ultrasonic device, or when the ultrasonic device is built in the microbubble generator previously used, the same device may also be used. Preferably, O2B-750S (built-in ultrasonic generator) manufactured by O2 Bubble company can be mentioned.

With such an ultrasonic device, fine bubbles having a size of several nanometers to several hundred nanometers can be produced inside the monomer composition or the aqueous solution. Further, the previously introduced inorganic fine particles are attached to around microbubbles, and the generated microbubbles can be stably maintained during the polymerization process described later. Therefore, it is useful for forming the porous structure of the super absorbent polymer and the gel strength can also be maintained at a constant level or higher.

Meanwhile, after microbubbles are formed in the monomer composition either by Process A or Process B or combined thereof, the monomer composition is subjected to crosslinking polymerization to form a hydrogel polymer.

For both Processes A and B, the formation of hydrogel polymer through crosslinking polymerization of a monomer mixture may be carried out by a conventional polymerization method. However, in order to proceed polymerization while stably maintaining bubbles in the monomer mixture formed by the above-mentioned methods (i.e., to form a polymer having a more developed porous structure), it is more preferable that the crosslinking polymerization is performed by (aqueous) solution polymerization.

Further, the polymerization process may be largely classified into a thermal polymerization and a photo-polymerization depending on a polymerization energy source. The thermal polymerization may be performed in a reactor like a kneader equipped with agitating spindles, and the photo-polymerization can be carried out in a reactor equipped with a movable conveyor belt.

As an example, the monomer mixture is injected into a reactor like a kneader equipped with the agitating spindles, and thermal polymerization is performed by providing hot air thereto or heating the reactor in order to obtain the hydrogel polymer. In this case, the hydrogel polymer, which is discharged from the outlet of the reactor according to the type of agitating spindles equipped in the reactor, can be obtained into a particle having several millimeters to several centimeters. Specifically, the resulting hydrogel polymer may be obtained in various forms according to the concentration of the monomer mixture injected thereto, the injection speed, or the like, and a hydrogel polymer having a (weight average) particle size of 2 to 50 mm may be generally obtained.

As another example, when the photo-polymerization of the monomer mixture is carried out in a reactor equipped with a movable conveyor belt, the hydrogel polymer may be obtained as a sheet. In this case, the thickness of the sheet may vary according to the concentration of the monomer mixture injected thereto and the injection speed. Usually, the polymer sheet is preferably controlled to have a thickness of 0.5 cm to 5 cm in order to uniformly polymerize the entire sheet and also secure production speed.

In this case, the hydrogel polymer obtained by the above-mentioned method may have a water content of 40 to 80% by weight. Meanwhile, the “water content” as used herein means a weight occupied by moisture with respect to a total amount of the hydrogel polymer, which may be the value obtained by subtracting the weight of the dried polymer from the weight of the hydrogel polymer. Specifically, the water content can be defined as a value calculated by measuring the weight loss due to evaporation of water in the polymer during the drying process of increasing the temperature of the polymer with infrared heating. At this time, the water content is measured under the drying conditions determined as follows: the drying temperature is increased from room temperature to about 180° C., and then the temperature is maintained at 180° C., and the total drying time is set as 20 minutes, including 5 minutes for the temperature rising step.

On the other hand, after the hydrogel polymer is prepared by the above-mentioned methods, the step of drying and pulverizing the hydrogel polymer may be carried out. Prior to such drying, the step of coarsely pulverizing the hydrogel polymer to produce a hydrogel polymer having a small average particle size may be first carried out.

In this coarse pulverization step, the hydrogel polymer may be pulverized into a size of 1.0 mm to 2.0 mm.

A pulverizing machine used in the coarse pulverization is not limited by its configuration, and specific examples thereof may include any one selected from a vertical pulverizer, a turbo cutter, a turbo grinder, a rotary cutter mill, a cutter mill, a disc mill, a shred crusher, a crusher, a chopper, and a disc cutter. However, it is not limited to the above-described examples.

Further, for the efficiency of the coarse pulverization, the coarse pulverization can be carried out multiple times depending on the size of the particle size. For example, the hydrogel polymer is subjected to a primary coarse pulverization into an average particle size of about 10 mm, again to a secondary coarse pulverization into an average particle size of about 5 mm, and then a third coarse pulverization into the above-mentioned particle size.

On the other hand, after the optional coarse pulverization, the hydrogel polymer can be dried. This drying temperature may be 50 to 250° C. When the drying temperature is less than 50° C., it is likely that the drying time becomes too long which will deteriorate the physical properties of the super absorbent polymer. When the drying temperature is higher than 250° C., only the surface of the polymer is excessively dried, which may cause fine powder generation, and the physical properties of the super absorbent polymer may be deteriorated. The drying may be carried out preferably at a temperature of 150 to 200° C., still more preferably at a temperature of 160 to 190° C. Meanwhile, the drying time may be 20 minutes to 15 hours, in consideration of the process efficiency and the like, but it is not limited thereto.

The drying method may be selected and used without being limited by its constitution if it is a method generally used for the above drying step. Specifically, the drying step may be carried out by methods such as hot air supply, infrared irradiation, microwave irradiation or ultraviolet irradiation. After the drying step as above is carried out, the water content of the polymer may be 0.05 to 10% by weight.

Next, the step of (finely) pulverizing the dried polymer obtained through such a drying step is carried out.

The polymer powder obtained after the pulverization step may have a particle size of 150 to 850 μm. Specific examples of a pulverizing device that can be used to pulverize into the above particle size may include a ball mill, a pin mill, a hammer mill, a screw mill, a roll mill, a disc mill, a jog mill or the like, but the present invention is not limited to the above-described example.

Then, in order to control the physical properties of the super absorbent polymer powder finally commercialized after the pulverization step, a separate step of classifying the polymer powder obtained after the pulverization depending on the particle size may be performed.

This classifying step may be carried out, for example, by a method of separating normal particles having a particle size of 150 to 850 μm and fine particles or macroparticles which fall outside such particle size range.

This classifying step may be carried out using a standard sieve according to a general method of classifying a super absorbent polymer.

The base polymer powder having such a particle size, that is, a particle size of 150 to 850 μm, may be commercialized through a surface crosslinking reaction step described hereinafter.

On the other hand, after progressing up to the classification described above, a super absorbent polymer may be produced by performing a step of crosslinking the surface of the base polymer powder, that is, by heat-treating and surface-crosslinking the base polymer powder in the presence of a surface crosslinking solution containing a surface crosslinking agent.

Here, the kind of the surface crosslinking agent contained in the surface crosslinking solution is not particularly limited. As a non-limiting example, the surface crosslinking agent may be one or more compounds selected from ethylene glycol diglycidyl ether, polyethylene glycol diglycidyl ether, glycerol polyglycidyl ether, propylene glycol diglycidyl ether, polypropylene glycol diglycidyl ether, ethylene carbonate, ethylene glycol, diethylene glycol, propylene glycol, triethylene glycol, tetraethylene glycol, propanediol, dipropylene glycol, polypropylene glycol, glycerin, polyglycerin, butanediol, heptanediol, hexanediol trimethylol propane, pentaerythritol, sorbitol, calcium hydroxide, magnesium hydroxide, aluminum hydroxide, iron hydroxide, calcium chloride, magnesium chloride, aluminum chloride, and iron chloride.

In this case, the content of the surface crosslinking agent may be appropriately controlled depending on the kind thereof, reaction conditions, and the like. Preferably, it may be controlled to 0.001 to 5 parts by weight based on 100 parts by weight of the base polymer powder. When the content of the surface crosslinking agent is too low, the surface crosslinking is not properly introduced, and the physical properties of the final superabsorbent polymer may be deteriorated. On the contrary, when the surface crosslinking agent is used in an excessive amount, the absorption capacity of the superabsorbent polymer may be rather lowered due to excessive surface crosslinking reaction, which is not preferable.

Further, the surface crosslinking solution may further include one or more solvents selected from water, ethanol, ethyleneglycol, diethyleneglycol, triethyleneglycol, 1,4-butanediol, propyleneglycol, ethyleneglycol monobutyl ether, propyleneglycol monomethyl ether, propyleneglycol monomethyl ether acetate, methylethylketone, acetone, methylamylketone, cyclohexanone, cyclopentanone, diethyleneglycol monomethyl ether, diethyleneglycol ethyl ether, toluene, xylene, butyrolactone, carbitol, methylcellosolve acetate and N,N-dimethylacetamide. The solvent may be contained in an amount of 0.5 to 10 parts by weight based on 100 parts by weight of the base polymer.

Further, the surface crosslinking solution may further include a thickener. When the surface of the base polymer powder is further crosslinked in the presence of the thickener in this way, deterioration of the physical properties may be minimized even after pulverization. Specifically, one or more selected from polysaccharides and polymers containing hydroxyl groups are used as the thickener. As the polysaccharides, gum-based thickeners and cellulose-based thickeners may be used. Specific examples of the gum-based thickeners may include xanthan gum, arabic gum, karaya gum, tragacanth gum, ghatti gum, guar gum, locust bean gum, psyllium seed gum, etc., and specific examples of the cellulose-based thickeners may include hydroxypropylmethylcellulose, carboxymethylcellulose, methylcellulose, hydroxymethylcellulose, hydroxyethylcellulose, hydroxypropylcellulose, hydroxyethylmethylcellulose, hydroxymethylpropylcellulose, hydroxyethylhydroxypropylcellulose, ethylhydroxyethylcellulose, methylhydroxypropylcellulose, etc. Meanwhile, specific examples of the polymers containing hydroxyl groups may include polyethylene glycol, polyvinyl alcohol, etc.

On the other hand, in order to perform the surface crosslinking, a method of adding and mixing the surface crosslinking solution and the base polymer in a reaction tank, a method of spraying a surface crosslinking solution onto the base polymer, a method of continuously providing and mixing the base polymer and the surface crosslinking solution to a continuously operating mixer, or the like may be used.

The surface crosslinking may be performed at a temperature of 100 to 250° C., and may be continuously performed after the drying and pulverizing steps proceeding at a relatively high temperature. At this time, the surface crosslinking reaction may be carried out for 1 to 120 minutes, or 1 to 100 minutes, or 10 to 60 minutes. In other words, in order to prevent the polymer particles from being damaged during the excessive reaction and thus the deterioration of the physical properties, while inducing the surface crosslinking reaction at the minimum, it may be carried out under the conditions of the surface crosslinking reaction described above.

As the superabsorbent polymer composition prepared as described above has a highly developed porous structure, it may exhibit improved absorption rate, and other various physical properties which provide advantageous properties.

The superabsorbent polymer formed by Process A or Process B that forms the particulate superabsorbent polymer composition of the invention may exhibit a greatly improved absorption rate, which is defined as a vortex absorption rate of 35 seconds or less, or 30 seconds or less, or 26 seconds or less, or 22 seconds or less, or 20 seconds or less, and 5 seconds or more, or 8 seconds or more, or 10 seconds or more. Furthermore, as the super absorbent polymer is produced by reducing the use amount of a foaming agent and/or a surfactant, excellent surface tension and bulk density may be maintained.

In the superabsorbent polymer, the absorption rate may be confirmed by a method of measuring the time (unit: second) required for the liquid vortex to disappear due to quick absorption when adding the super absorbent resin to a physiological saline solution and stirring it. The bulk density and surface tension can be measured according to the method described in Examples provided hereinafter.

The particulate superabsorbent polymer composition has a particle size range of 150 to 850 min. Particles having a particle size of 600 μm or more may be contained in an amount of 12 wt % or less, or 10 wt % or less of the particulate superabsorbent composition. Further, the particles having a particle size of 300 μm or less may be contained in an amount of 20 wt % or less, or 15 wt % or less.

As the particulate superabsorbent polymer composition has a relatively uniform particle size distribution, the composition may exhibit excellent and uniform absorption characteristics.

Further, the particulate superabsorbent polymer composition may have a centrifuge retention capacity (CRC) of 25 to 35 g/g, or 28 to 34 g/g, or 29 to 33 g/g, as measured according to EDANA recommended test method WSP 241.3. Such centrifuge retention capacity can reflect the excellent absorption capacity of the composition.

Further, the particulate superabsorbent polymer composition may have an absorbency under load (AUL) of 14 to 23 g/g, or 18 to 21 g/g at 0.9 psi as measured according to EDANA recommended test method WSP 242.3. By satisfying these ranges, the particulate superabsorbent polymer composition may exhibit excellent absorption capacity and moisture retaining properties even under load.

Further, the particulate superabsorbent polymer composition may exhibit characteristics that a gel bed permeability (GBP) is 25 to 50 darcy, or 30 to 48 darcy, or 35 to 45 darcy, and thereby excellent liquid permeability can be exhibited.

Hereinafter, preferred examples and test methods are presented to aid in understanding of the invention. However, the examples are for illustrative purposes only, and the scope of the invention is not intended to be limited thereby.

### Example 1: Preparation of Particulate Superabsorbent Polymer Composition

8.6 g (80 ppmw based on the monomer) of 0.5 wt % IRGACURE 819 initiator diluted with acrylic acid and 12.3 g of 20 wt % polyethylene glycol diacrylate (PEGDA, Mw=400) diluted with acrylic acid were mixed to prepare a solution (solution A).

540 g of acrylic acid and the solution A were injected into a 2 L-volume glass reactor surrounded by a jacket through which a heating medium pre-cooled at 25° 0 was circulated.

Then, to the glass reactor, 832 g of 25 wt % caustic soda solution (solution C) was slowly added dropwise and mixed. After confirming that the temperature of the mixed solution increased to about 72° C. or higher by neutralization heat, the mixed solution was left until it was cooled. A neutralization degree of acrylic acid in the mixed solution thus obtained was about 70 mol %.

On the other hand, as a surfactant, a solution containing sodium dodecylsulfate (HLB: about 40) diluted with water and SPAN-80 (HLB: 4.6) was converted to a solution D containing bubbles using a microbubble machine (OB-750S, manufactured by O2 Bubble) circulating at a flow rate of 500 kg/h. In addition, 30 g of 4 wt % sodium persulfate solution (solution E) diluted with water was prepared. Then, when the temperature of the mixed solution was cooled to about 45° C., solutions D and E previously prepared were added to the mixed solution and mixed. At this time, the content of sodium dodecyl sulfate in the solution D was adjusted to 110 ppmw relative to acrylic acid, and SPAN-80 to 50 ppmw so that the total amount of the surfactant was 160 ppmw.

Then, the above-prepared mixed solution was poured in a Vat-type tray (15 cm in width×15 cm in length) installed in a square polymerizer which had a light irradiation device installed at the top and was preheated to 80° C. The mixed solution was then subjected to light irradiation. It was confirmed that at about 20 seconds after light irradiation, gel was formed from the surface, and that at about 30 seconds after light irradiation, polymerization occurred concurrently with forming. Then, the polymerization reaction was allowed for additional 2 minutes, and the polymerized sheet was taken and cut into a size of 3 cm×3 cm.

Then, it was subjected to a chopping process using a meat chopper to prepare the cut sheet as crumbs. The average particle size of the prepared crumbs was 1.5 mm.

Then, the crumbs were dried in an oven capable of shifting airflow up and down. The crumbs were uniformly dried by flowing hot air at 180° C. from the bottom to the top for 15 minutes and from the top to the bottom for 15 minutes such that the dried crumbs had a water content of about 2 wt % or less. The dried crumbs were pulverized using a pulverizer and classified, and a base polymer having a size of 150 to 850 μm was obtained.

Subsequently, 100 g of the above-prepared base polymer was mixed with a crosslinking agent solution which was obtained by mixing 4.5 g of water, 1 g of ethylene carbonate, 0.05 g of Aerosil 200 (EVONIK) and 0.25 g of 20 wt % water-dispersed silica (Snowtex, ST-O) solution, and then surface crosslinking reaction was performed at 190° C. for 30 minutes. The resulting product was pulverized and then passed through a sieve to obtain a surface-crosslinked super absorbent polymer having a particle size of 150 to 850 μm. 0.1 g of Aerosil 200 was further mixed with the obtained super absorbent by a dry method to prepare a super absorbent polymer.

### Example 2: Preparation of Particulate Superabsorbent Polymer Composition

A composition was prepared in the same manner as in Example 1, except that only anionic surfactant sodium dodecyl sulfate was used without using a nonionic surfactant SPAN-80, and the content thereof was adjusted to be 80 ppmw relative to acrylic acid.

### Example 3: Preparation of Particulate Superabsorbent Polymer Composition

A composition was prepared in the same manner as in Example 1, except that only an anionic surfactant sodium dodecyl sulfate was used without using a nonionic surfactant SPAN-80, the content thereof was adjusted to be 160 ppmw relative to acrylic acid, and the finally obtained composition was subjected to a water treatment so as to adjust the water content in the product to about 2% by weight.

### Example 4: Preparation of Particulate Superabsorbent Polymer Composition

A composition was prepared in the same manner as in Example 1, except that the content of sodium dodecyl sulfate was adjusted to 50 ppmw relative to acrylic acid, and the content of SPAN-80 was adjusted to 250 ppmw relative to acrylic acid.

### Example 5: Preparation of Particulate Superabsorbent Polymer Composition

A super absorbent polymer was prepared in the same manner as in Example 1, except that the content of sodium dodecyl sulfate was adjusted to 150 ppmw relative to acrylic acid and the content of TWEEN 80 (HLB: 15) was adjusted to 30 ppmw relative to acrylic acid.

### Example 6: Preparation of Particulate Superabsorbent Polymer Composition

8.6 g (80 ppmw based on the monomer) of 0.5 wt % IRGACURE 819 initiator diluted with acrylic acid and 12.3 g of 20 wt % polyethylene glycol diacrylate (PEGDA, Mw=400) diluted with acrylic acid were mixed to prepare a solution (solution A).

540 g of acrylic acid and the solution A were injected into a 2 L-volume glass reactor surrounded by a jacket through which a heating medium pre-cooled at 25° C. was circulated.

Then, to the glass reactor, 832 g of 25 wt % caustic soda solution (solution C) was slowly added dropwise and mixed. After confirming that the temperature of the mixed solution increased to about 72° C. or higher by neutralization heat, the mixed solution was left until it was cooled. A neutralization degree of acrylic acid in the mixed solution thus obtained was about 70 mol %.

Then, water was added to a microbubble machine (OB-750S, manufactured by O2 Bubble) circulating at a flow rate of 500 kg/h to prepare a solution D in which bubbles were generated. Silica was added thereto, and the solution was put into an ultrasonic device (OB-750S, manufactured by O2 Bubble) to prepare a solution F. When the temperature of the neutralized mixed solution was cooled to about 45° C., the solution F previously prepared were added to the mixed solution and mixed. At this time, silica was added in an amount of 0.05 part by weight based on 100 parts by weight of the mixed solution.

Then, the above-prepared mixed solution was poured in a Vat-type tray (15 cm in width×15 cm in length) installed in a square polymerizer which had a light irradiation device installed at the top and was preheated to 80° C. The mixed solution was then subjected to light irradiation. It was confirmed that at about 20 seconds after light irradiation, gel was formed from the surface, and that at about 30 seconds after light irradiation, polymerization occurred concurrently with forming. Then, the polymerization reaction was performed for additional 2 minutes, and the polymerized sheet was taken and cut into a size of 3 cm×3 cm.

Then, it was subjected to a chopping process using a meat chopper to prepare the cut sheet as crumbs. The average particle size of the prepared crumbs was 1.5 mm.

Then, the crumbs were dried in an oven capable of shifting airflow up and down. The crumbs were uniformly dried by flowing hot air at 180° C. from the bottom to the top for 15 minutes and from the top to the bottom for 15 minutes such that the dried crumbs had a water content of about 2 wt % or less. The dried crumbs were pulverized using a pulverizer and classified, and a base polymer having a size of 150 to 850 m was obtained.

Subsequently, 100 g of the above-prepared base polymer was mixed with a crosslinking agent solution which was obtained by mixing 4.5 g of water, 1 g of ethylene carbonate, 0.05 g of Aerosil 200 (EVONIK) and 0.25 g of 20 wt % water-dispersed silica (Snowtex, ST-O) solution, and then surface crosslinking reaction was performed at 190° C. for 30 minutes. The resulting product was pulverized and then passed through a sieve to obtain a surface-crosslinked super absorbent polymer having a particle size of 150 to 850 m. 0.1 g of Aerosil 200 was further mixed with the obtained super absorbent by a dry method to prepare a super absorbent polymer.

### Example 7: Preparation of Particulate Superabsorbent Polymer Composition

The same procedure as in Example 1 was repeated until the neutralization solution was produced in Example 6.

Separately, an aqueous solution containing sodium dodecylsulfate diluted with water was added to a microbubble machine (OB-750S, manufactured by O2 Bubble) circulating at a flow rate of 500 kg/h to prepare a solution D in which bubbles were generated. At this time, the content of sodium dodecylsulfate in the solution D was adjusted to be 10 ppmw based on the total weight of the acrylic acid. Silica was added thereto, and the solution was put into an ultrasonic device (OB-750S, manufactured by O2 Bubble) to prepare a solution F. When the temperature of the neutralized mixed solution was cooled to about 45° C., the solution F previously prepared were added to the mixed solution and mixed. At this time, silica was added in an amount of 0.05 part by weight based on 100 parts by weight of the acrylic acid.

The subsequent procedures were performed in the same manner as in Example 1 to prepare a super absorbent polymer.

### Example 8: Preparation of Particulate Superabsorbent Polymer Composition

A super absorbent polymer was prepared in the same manner as in Example 7, except that the content of sodium dodecylsulfate in the solution D was adjusted to 50 ppmw based on the total weight of the acrylic acid.

### Example 9: Preparation of Particulate Superabsorbent Polymer Composition

A super absorbent polymer was prepared in the same manner as in Example 7, except that the content of sodium dodecylsulfate in the solution D was adjusted to 100 ppmw based on the total weight of the acrylic acid.

### Comparative Example 1: Preparation of Super Absorbent Polymer

8.6 g (80 ppmw based on the monomer) of 0.5 wt % IRGACURE 819 initiator diluted with acrylic acid and 12.3 g of 20 wt % polyethylene glycol diacrylate (PEGDA, Mw=400) diluted with acrylic acid were mixed to prepare a solution (solution A).

540 g of acrylic acid and the solution A were injected into a 2 L-volume glass reactor surrounded by a jacket through which a heating medium pre-cooled at 25° C. was circulated.

Then, to the glass reactor, 832 g of 25 wt % caustic soda solution (solution C) was slowly added dropwise and mixed. After confirming that the temperature of the mixed solution increased to about 72° C. or higher by neutralization heat, the mixed solution was left until it was cooled. A neutralization degree of acrylic acid in the mixed solution thus obtained was about 70 mol %.

On the other hand, as a surfactant, a solution D containing sodium dodecylsulfate (HLB: about 40) diluted with water and SPAN-80 (HLB: 4.6) was prepared. In addition, 30 g of 4 wt % sodium persulfate solution (solution E) diluted with water was prepared. Then, when the temperature of the mixed solution was cooled to about 45° C., solutions D and E previously prepared were added to the mixed solution and mixed. At this time, the content of sodium dodecyl sulfate in the solution D was adjusted to 110 ppmw relative to acrylic acid, and SPAN-80 to 50 ppmw so that the total amount of the surfactant was 160 ppmw.

Then, the above-prepared mixed solution was poured in a Vat-type tray (15 cm in width×15 cm in length) installed in a square polymerizer which had a light irradiation device installed at the top and was preheated to 80° C. The mixed solution was then subjected to light irradiation. It was confirmed that at about 20 seconds after light irradiation, gel was formed from the surface, and that at about 30 seconds after light irradiation, polymerization occurred concurrently with forming. Then, the polymerization reaction was performed for additional 2 minutes, and the polymerized sheet was taken and cut in a size of 3 cm×3 cm.

Then, it was subjected to a chopping process using a meat chopper to prepare the cut sheet as crumbs. The average particle size of the prepared crumbs was 1.5 mm.

Then, the crumbs were dried in an oven capable of shifting airflow up and down. The crumbs were uniformly dried by flowing hot air at 180° C. from the bottom to the top for 15 minutes and from the top to the bottom for 15 minutes such that the dried crumbs had a water content of about 2 wt % or less. The dried crumbs were pulverized using a pulverizer and classified by size, and a base polymer having a size of 150 to 850 μm was obtained.

Subsequently, 100 g of the above-prepared base polymer was mixed with a crosslinking agent solution which was obtained by mixing 4.5 g of water, 1 g of ethylene carbonate, 0.05 g of Aerosil 200 (EVONIK) and 0.25 g of 20 wt % water-dispersed silica (Snowtex, ST-O) solution, and then surface crosslinking reaction was performed at 190° C. for 30 minutes. The resulting product was pulverized and then passed through a sieve to obtain a surface-crosslinked super absorbent polymer having a particle size of 150 to 850 μm. 0.1 g of Aerosil 200 was further mixed with the obtained super absorbent by a dry method.

### Comparative Example 2: Preparation of Super Absorbent Polymer

A super absorbent polymer was prepared in the same manner as in Comparative Example 1, except that the content of sodium dodecyl sulfate in the solution D was adjusted to 350 ppmw relative to acrylic acid, and SPAN-80 to 50 ppmw so that the total amount of surfactant was 400 ppmw.

### Comparative Example 3: Preparation of Super Absorbent Polymer

A super absorbent polymer was prepared in the same manner as in Example 1, except that only an anionic surfactant sodium dodecyl sulfate was used without using a nonionic surfactant SPAN-80, and the content thereof was adjusted to be 400 ppmw relative to acrylic acid.

### Comparative Example 4: Preparation of Super Absorbent Polymer

8.6 g (80 ppmw based on the monomer) of 0.5 wt % IRGACURE 819 initiator diluted with acrylic acid and 12.3 g of 20 wt % polyethylene glycol diacrylate (PEGDA, Mw=400) diluted with acrylic acid were mixed to prepare a solution (solution A).

540 g of acrylic acid and the solution A were injected into a 2 L-volume glass reactor surrounded by a jacket through which a heating medium pre-cooled at 25° C. was circulated.

Then, to the glass reactor, 832 g of 25 wt % caustic soda solution (solution C) was slowly added dropwise and mixed. After confirming that the temperature of the mixed solution increased to about 72° C. or higher by neutralization heat, the mixed solution was left until it was cooled. A neutralization degree of acrylic acid in the mixed solution thus obtained was about 70 mol %.

On the other hand, as a surfactant, a solution D-1 containing sodium dodecylsulfate diluted with water and a solution D-2 containing 4 wt % sodium dicarbonate were prepared, respectively. In addition, 30 g of 4 wt % sodium persulfate solution (solution E) diluted with water was prepared. Then, when the temperature of the mixed solution was cooled to about 45° C., solutions D-1, D-2 and E previously prepared were added to the mixed solution and mixed. At this time, the content of sodium dodecyl sulfate in the solution D-1 was adjusted to be 200 ppmw relative to acrylic acid.

Then, the above-prepared mixed solution was poured in a Vat-type tray (15 cm in width×15 cm in length) installed in a square polymerizer which had a light irradiation device installed at the top and was preheated to 80° C. The mixed solution was then subjected to light irradiation. It was confirmed that at about 20 seconds after light irradiation, gel was formed from the surface, and that at about 30 seconds after light irradiation, polymerization occurred concurrently with forming. Then, the polymerization reaction was performed for additional 2 minutes, and the polymerized sheet was taken and cut in a size of 3 cm×3 cm.

Then, it was subjected to a chopping process using a meat chopper to prepare the cut sheet as crumbs. The average particle size of the prepared crumbs was 1.5 mm.

Then, the crumbs were dried in an oven capable of shifting airflow up and down. The crumbs were uniformly dried by flowing hot air at 180° C. from the bottom to the top for 15 minutes and from the top to the bottom for 15 minutes such that the dried crumbs had a water content of about 2 wt % or less. The dried crumbs were pulverized using a pulverizer and classified by size, and a base polymer having a size of 150 to 850 μm was obtained.

Subsequently, 100 g of the above-prepared base polymer was mixed with a crosslinking agent solution which was obtained by mixing 4.5 g of water, 1 g of ethylene carbonate, 0.05 g of Aerosil 200 (EVONIK) and 0.25 g of 20 wt % water-dispersed silica (Snowtex, ST-O) solution, and then surface crosslinking reaction was allowed at 190° C. for 30 minutes. The resulting product was pulverized and then passed through a sieve to obtain a surface-crosslinked super absorbent polymer having a particle size of 150 to 850 μm. 0.1 g of Aerosil 200 was further mixed with the obtained super absorbent by a dry method.

### Comparative Example 5: Preparation of Super Absorbent Polymer

A super absorbent polymer was prepared in the same manner as in Comparative Example 1, except that sodium dodecyl sulfate was only used in the solution D and adjusted to 200 ppmw relative to acrylic acid. In addition, 30 g of 4 wt % sodium bicarbonate diluted with water (solution E) was prepared.

### Comparative Example 6: Preparation of Super Absorbent Polymer

The same procedure as in Comparative Example 5 was repeated until the neutralization solution was produced in Comparative Example 5.

Separately, an aqueous solution containing sodium dodecylsulfate diluted with water was added to a microbubble machine (OB-750S, manufactured by O2 Bubble) circulating at a flow rate of 500 kg/h to prepare a solution D in which bubbles were generated. At this time, the content of sodium dodecylsulfate in the solution D was adjusted to 10 ppmw based on the total weight of the acrylic acid. When the temperature of the neutralized mixed solution was cooled to about 45° C., the solution D previously prepared were added to the mixed solution and mixed.

The subsequent procedures were performed in the same manner as in Comparative Example 1 to prepare a super absorbent polymer.

### Comparative Example 7: Preparation of Super Absorbent Polymer

A super absorbent polymer was prepared in the same manner as in Comparative Example 6, except that silica was added to the solution D in an amount of 0.05 part by weight based on 100 parts by weight of the acrylic acid in Comparative Example 6.

### Comparative Example 8: Preparation of Super Absorbent Polymer

The same procedure as in Comparative Example 5 was repeated until the neutralization solution was produced in Comparative Example 5.

Separately, an aqueous solution containing sodium dodecylsulfate diluted with water was added to a microbubble machine (OB-750S, manufactured by O2 Bubble) circulating at a flow rate of 500 kg/h to prepare a solution D in which bubbles were generated. At this time, the content of sodium dodecylsulfate in the solution D was adjusted to be 200 ppmw based on the total weight of the acrylic acid. The solution D was put into an ultrasonic device (OB-750S, manufactured by O2 Bubble) to prepare a solution F. When the temperature of the mixed solution was cooled to about 45° C., the solution F previously prepared were added to the mixed solution and mixed.

The subsequent procedures were performed in the same manner as in Comparative Example 1 to prepare a super absorbent polymer.

### Test Methods for Evaluating Properties of Particulate Superabsorbent Polymer Compositions

The physical properties of the super absorbent polymers prepared in Examples and Comparative Examples were evaluated by the following methods, and the results are shown in Table 1 below.

### Bulk Density

About 100 g of the super absorbent polymer was placed in a funnel-shaped bulk density tester and flown down into a 100 ml container. Then, the weight of the super absorbent polymer contained in the container was measured. The bulk density was calculated as (super absorbent polymer weight)/(container volume, 100 ml). (unit: g/ml).

### Vortex Time

The Vortex Time is the amount of time in seconds required for a predetermined mass of superabsorbent particles to close a vortex created by stirring 50 milliliters of 0.9 percent by weight sodium chloride solution at 600 revolutions per minute on a magnetic stir plate. The time it takes for the vortex to close is an indication of the free swell absorbing rate of the particles. The vortex time test can be performed at a temperature is 23° C. and relative humidity of 50% according to the following procedure:
(1) Measure 50 milliliters (±0.01 milliliter) of 0.9 percent by weight sodium chloride solution into the 100-milliliter beaker.
(2) Place a 7.9 millimeters×32 millimeters TEFLON® covered magnetic stir bar without rings (such as that commercially available under the trade designation S/P® brand single pack round stirring bars with removable pivot ring) into the beaker.
(3) Program a magnetic stir plate (such as that commercially available under the trade designation DATAPLATE® Model #721) to 600 revolutions per minute.
(4) Place the beaker on the center of the magnetic stir plate such that the magnetic stir bar is activated. The bottom of the vortex should be near the top of the stir bar. The superabsorbent particles are pre-screened through a U.S. standard #30 mesh screen (0.595 millimeter openings) and retained on a U.S. standard #50 mesh screen (0.297 millimeter openings).
(5) Weigh out the required mass of the superabsorbent particles to be tested on weighing paper.
(6) While the sodium chloride solution is being stirred, quickly pour the absorbent polymer to be tested into the saline solution and start a stopwatch. The superabsorbent particles to be tested should be added to the saline solution between the center of the vortex and the side of the beaker.
(7) Stop the stopwatch when the surface of the saline solution becomes flat and record the time. The time, recorded in seconds, is reported as the vortex time.

### Centrifuge Retention Capacity (CRC)

The Centrifuge Retention Capacity (CRC) test measures the ability of superabsorbent particles to retain liquid after being saturated and subjected to centrifugation under controlled conditions. The resultant retention capacity is stated as grams of liquid retained per gram weight of the sample (g/g) and is measured according to EDANA recommended test method WSP 241.3. The sample to be tested is prepared from particles that are prescreened through a U.S. standard 30-mesh screen and retained on a U.S. standard 50-mesh screen. The particles can be prescreened by hand or automatically and are stored in a sealed airtight container until testing. The retention capacity is measured by placing 0.2±0.005 grams of the prescreened sample into a water-permeable bag that will contain the sample while allowing a test solution (0.9 weight percent sodium chloride in distilled water) to be freely absorbed by the sample. A heat-sealable tea bag material, such as model designation 1234T heat sealable filter paper, can be suitable. The bag is formed by folding a 5-inch by 3-inch sample of the bag material in half and heat-sealing two of the open edges to form a 2.5-inch by 3-inch rectangular pouch. The heat seals can be about 0.25 inches inside the edge of the material. After the sample is placed in the pouch, the remaining open edge of the pouch can also be heat-sealed. Empty bags can be made to serve as controls. Three samples (e.g., filled and sealed bags) are prepared for the test. The filled bags are tested within three minutes of preparation unless immediately placed in a sealed container, in which case the filled bags must be tested within thirty minutes of preparation.

The bags are placed between two TEFLON® coated fiberglass screens having 3-inch openings (Taconic Plastics, Inc., Petersburg, N.Y.) and submerged in a pan of the test solution at 23° C., making sure that the screens are held down until the bags are completely wetted. After wetting, the samples remain in the solution for about 30±1 minutes, at which time they are removed from the solution and temporarily laid on a non-absorbent flat surface. For multiple tests, the pan should be emptied and refilled with fresh test solution after 24 bags have been saturated in the pan.

The wet bags are then placed into the basket of a suitable centrifuge capable of subjecting the samples to a g-force of about 350. One suitable centrifuge is a Heraeus LaboFuge 400 having a water collection basket, a digital rpm gauge, and a machined drainage basket adapted to hold and drain the bag samples. Where multiple samples are centrifuged, the samples can be placed in opposing positions within the centrifuge to balance the basket when spinning. The bags (including the wet, empty bags) are centrifuged at about 1,600 rpm (e.g., to achieve a target g-force of about 350), for 3 minutes. The bags are removed and weighed, with the empty bags (controls) being weighed first, followed by the bags containing the samples. The amount of solution retained by the sample, taking into account the solution retained by the bag itself, is the centrifuge retention capacity (CRC) of the sample, expressed as grams of fluid per gram of sample. More particularly, the centrifuge retention capacity is determined as:

Sample Bag Weight After Centrifuge−Empty Bag Weight After Centrifuge−Dry Sample Weight/Dry Sample Weight

The three samples were tested and the results were averaged to determine the retention capacity (CRC) of the superabsorbent material. The samples were tested at 23° C. and 50% relative humidity.

### Absorbent Capacity

The absorbent capacity of superabsorbent particles can be measured using an Absorbency Under Load (“AUL”) test, which is a well-known test for measuring the ability of superabsorbent particles to absorb a 0.9 wt. % solution of sodium chloride in distilled water at room temperature (test solution) while the material is under a load. For example, 0.16 grams of superabsorbent particles can be confined within a 5.07 cm² area of an Absorbency Under Load (“AUL”) cylinder under a nominal pressure of 0.01 psi, 0.3 psi, or 0.9 psi. The sample is allowed to absorb the test solution from a dish containing excess fluid. At predetermined time intervals, a sample is weighed after a vacuum apparatus has removed any excess interstitial fluid within the cylinder. This weight versus time data is then used to determine the Absorption Rates at various time intervals.

The AUL test apparatus is measured according to EDANA recommended test method WSP 242.3 which is similar to a GATS (gravimetric absorbency test system), available from M/K Systems, as well as the system described by Lichstein at pages 129-142 of the INDA Technological Symposium Proceedings, March 1974. A ported disk is also utilized having ports confined within a 2.5-centimeter diameter area. The resultant AUL is stated as grams of liquid retained per gram weight of the sample (g/g).

To carry out the test, the following steps may be performed:
(1) Wipe the inside of the AUL cylinder with an anti-static cloth, and weigh the cylinder, weight and piston;
(2) Record the weight as CONTAINER WEIGHT in grams to the nearest milligram;
(3) Slowly pour the 0.16±0.005 gram sample of the superabsorbent particles into the cylinder so that the particles do not make contact with the sides of the cylinder or it can adhere to the walls of the AUL cylinder;
(4) Weigh the cylinder, weight, piston, and superabsorbent particles and record the value on the balance, as DRY WEIGHT in grams to the nearest milligram;
(5) Gently tap the AUL cylinder until the superabsorbent particles are evenly distributed on the bottom of the cylinder;
(6) Gently place the piston and weight into the cylinder;
(7) Place the test fluid (0.9 wt. % aqueous sodium chloride solution) in a fluid bath with a large mesh screen on the bottom;
(8) Simultaneously start the timer and place the superabsorbent particles and cylinder assembly onto the screen in the fluid bath for an hour. The level in the bath should be at a height to provide at least a 1 cm positive head above the base of the cylinder;
(9) Gently swirl the sample to release any trapped air and ensure the superabsorbent particles are in contact with the fluid.
(10) Remove the cylinder from the fluid bath at a designated time interval and immediately place the cylinder on the vacuum apparatus (ported disk on the top of the AUL chamber) and remove excess interstitial fluid for 10 seconds;
(11) Wipe the exterior of the cylinder with paper toweling or tissue;
(12) Weigh the AUL assembly (i.e., cylinder, piston and weight), with the superabsorbent particles and any absorbed test fluid immediately and record the weight as WET WEIGHT in grams to the nearest milligram and the time interval; and

The “absorbent capacity” of the superabsorbent particles at a designated time interval is calculated in grams liquid by grams superabsorbent by the following formula:

(Wet Weight−Dry Weight)/(Dry Weight−Container Weight)

### Surface Tension (S/T)

The surface tension of the liquid was measured using a Fisher Surface Tensiometer. The measurement method was as follows. About 150 g of 0.9 wt % saline solution was placed in a 250 mL beaker, and a 2 inch deep vortex was created while stirring with a magnetic stirrer.

Then 1.0±0.01 g of sample was weighed and placed in the stirring solution. When the stirring time exceeded 3 minutes, stirring was stopped, and a stirring rod was removed with clean tweezers, and then the sample was left for at least 15 minutes so as to allow a gel of the sample to settle to the bottom. After leaving for 15 minutes, the tip of the pipette was inserted directly beneath the surface of the test liquid to withdraw sufficient solution.

The test liquid was transferred to the clean sample cup. The sample cup containing the test liquid was placed on the sample table and then the dial was adjusted to zero.

A clean platinum-iridium ring (P-I Ring) was fixed to a tension meter with calibration. The sample table was lifted up by turning a bottom knob in a clockwise direction until it was submerged under the surface of the test liquid of P-I ring.

The P-I ring was immersed for about 35 seconds, and then the rotating pin was loosened to hang freely. The bottom knob was turned until the reference arm was parallel to the line above the mirror. The P-I ring was slowly lifted up at a constant rate.

The scale of dials on the front was recorded when leaving the surface of the test liquid of P-I ring. This is the surface tension expressed by dyne/m³. The actual surface tension value is calculated by correcting the measured surface tension value.

$$\begin{matrix}Surface \\ Tension\end{matrix} ( s ) = P \times ( 0.7250 \sqrt{\frac{0.01452 \times F}{{5.930}^{*}}} + 0.04534 - \frac{1.679}{53.1218} )$$

$$Actual surface tension = P \times F 20$$

$$P = measured surface tension ( scale read from dial )$$

$$F = adjusted equation below$$

$$\text{?} = 0.7250 + \sqrt{\frac{0.01452 \times F}{{C}^{2}}} + 0.04534 - \frac{1.679 r}{R}$$

$$R = radius of the ring$$

$$r = radius of the ring bar$$

$$C = circumference of ring$$

$$\text{?} \text{indicates text missing or illegible when filed}$$

### Free-Swell Gel Bed Permeability (GBP) Test

As used herein, the Free Swell Gel Bed Permeability (GBP) Test determines the permeability of a swollen bed of superabsorbent material under what is commonly referred to as “free swell” conditions. The term “free swell” means that the superabsorbent material is allowed to swell without a swell restraining load upon absorbing test solution as will be described. This test is described in U.S. Patent Publication No. 2010/0261812 to Qin, which is incorporated herein by reference thereto. For instance, a test apparatus can be employed that contains a sample container and a piston, which can include a cylindrical LEXAN shaft having a concentric cylindrical hole bored down the longitudinal axis of the shaft. Both ends of the shaft can be machined to provide upper and lower ends. A weight can rest on one end that has a cylindrical hole bored through at least a portion of its center. A circular piston head can be positioned on the other end and provided with a concentric inner ring of seven holes, each having a diameter of about 0.95 cm, and a concentric outer ring of fourteen holes, each having a diameter of about 0.95 cm. The holes are bored from the top to the bottom of the piston head. The bottom of the piston head can also be covered with a biaxially stretched mesh stainless steel screen. The sample container can contain a cylinder and a 100-mesh stainless steel cloth screen that is biaxially stretched to tautness and attached to the lower end of the cylinder. Superabsorbent particles can be supported on the screen within the cylinder during testing.

The cylinder can be bored from a transparent LEXAN rod or equivalent material, or it can be cut from a LEXAN tubing or equivalent material, and has an inner diameter of about 6 cm (e.g., a cross-sectional area of about 28.27 cm²), a wall thickness of about 0.5 cm and a height of approximately 5 cm. Drainage holes can be formed in the sidewall of the cylinder at a height of approximately 4.0 cm above the screen to allow liquid to drain from the cylinder to thereby maintain a fluid level in the sample container at approximately 4.0 cm above the screen. The piston head can be machined from a LEXAN rod or equivalent material and has a height of approximately 16 mm and a diameter sized such that it fits within the cylinder with minimum wall clearance but still slides freely. The shaft can be machined from a LEXAN rod or equivalent material and has an outer diameter of about 2.22 cm and an inner diameter of about 0.64 cm. The shaft upper end is approximately 2.54 cm long and approximately 1.58 cm in diameter, forming an annular shoulder to support the annular weight. The annular weight, in turn, has an inner diameter of about 1.59 cm so that it slips onto the upper end of the shaft and rests on the annular shoulder formed thereon. The annular weight can be made from stainless steel or from other suitable materials resistant to corrosion in the presence of the test solution, which is 0.9 wt. % sodium chloride solution in distilled water. The combined weight of the piston and annular weight equals approximately 596 grams, which corresponds to a pressure applied to the sample of about 0.3 pounds per square inch, or about 20.7 dynes/cm², over a sample area of about 28.27 cm². When the test solution flows through the test apparatus during testing as described below, the sample container generally rests on a 16-mesh rigid stainless steel support screen. Alternatively, the sample container can rest on a support ring diametrically sized substantially the same as the cylinder so that the support ring does not restrict flow from the bottom of the container.

To conduct the Gel Bed Permeability Test under “free swell” conditions, the piston, with the weight seated thereon, is placed in an empty sample container and the height from the bottom of the weight to the top of the cylinder is measured using a caliper or suitable gauge accurate to 0.01 mm. The height of each sample container can be measured empty and which piston and weight is used can be tracked when using multiple test apparatus. The same piston and weight can be used for measurement when the sample is later swollen following saturation. The sample to be tested is prepared from superabsorbent particles that are prescreened through a U.S. standard 30-mesh screen and retained on a U.S. standard 50-mesh screen. The particles can be prescreened by hand or automatically. Approximately 0.9 grams of the sample is placed in the sample container, and the container, without the piston and weight therein, is then submerged in the test solution for a time period of about 60 minutes to saturate the sample and allow the sample to swell free of any restraining load. At the end of this period, the piston and weight assembly is placed on the saturated sample in the sample container and then the sample container, piston, weight, and sample are removed from the solution. The thickness of the saturated sample is determined by again measuring the height from the bottom of the weight to the top of the cylinder, using the same caliper or gauge used previously provided that the zero point is unchanged from the initial height measurement. The height measurement obtained from measuring the empty sample container, piston, and weight is subtracted from the height measurement obtained after saturating the sample. The resulting value is the thickness, or height “H” of the swollen sample.

The permeability measurement is initiated by delivering a flow of the test solution into the sample container with the saturated sample, piston, and weight inside. The flow rate of test solution into the container is adjusted to maintain a fluid height of about 4.0 cm above the bottom of the sample container. The quantity of solution passing through the sample versus time is measured gravimetrically. Data points are collected every second for at least twenty seconds once the fluid level has been stabilized to and maintained at about 4.0 cm in height. The flow rate Q through the swollen sample is determined in units of grams/second (g/s) by a linear least-square fit of fluid passing through the sample (in grams) versus time (in seconds). The permeability is obtained by the following equation:

K=(1.01325×10⁸)*[Q*H*Mu]/[A*Rho*P]

where

K=Permeability (darcys),

Q=flow rate (g/sec),

H=height of sample (cm),

Mu=liquid viscosity (poise) (approximately 1 centipoise for the test solution used with this test),

A=cross-sectional area for liquid flow (cm²),

Rho=liquid density (g/cm³) (approximately 1 g/cm³ for the test solution used with this Test), and

P=hydrostatic pressure (dynes/cm²) (normally approximately 3,923 dynes/cm²), which can be calculated from Rho*g*h, where Rho=liquid density (g/cm³), g=gravitational acceleration, nominally 981 cm/sec², and h=fluid height, e.g., 4.0 cm.

A minimum of three samples were tested and the results were averaged to determine the free swell gel bed permeability of the sample. The samples were tested at 23° C. and 50% relative humidity.

TABLE 1
[Physical Properties of Examples for Process
A or B versus Comparative Examples]

| | Bulk | | | | | Surface |
|---|---|---|---|---|---|---|
| | density | Vortex | CRC | 0.9 AUL | GBP | tension |
| | (g/ml) | (sec) | (g/g) | (g/g) | (darcy) | (mN/m) |
| Example 1 - | 0.57 | 23 | 30.8 | 18.7 | 45 | 68 |
| Process A | | | | | | |
| Example 2 - | 0.59 | 29 | 30.5 | 19.1 | 36 | 70 |
| Process A | | | | | | |
| Example 3 - | 0.61 | 25 | 30.1 | 18.2 | 42 | 66 |
| Process A | | | | | | |
| Example 4 - | 0.61 | 27 | 30.2 | 18.2 | 44 | 65 |
| Process A | | | | | | |
| Example 5 - | 0.60 | 24 | 29.7 | 19.4 | 29.7 | 66 |
| Process A | | | | | | |
| Example 6 - | 0.63 | 35 | 30 | 19.5 | 50 | 71 |
| Process B | | | | | | |
| Example 7 - | 0.57 | 25 | 30.2 | 18.3 | 47 | 70.8 |
| Process B | | | | | | |
| Example 8 - | 0.57 | 20 | 30.4 | 17.8 | 40 | 71 |
| Process B | | | | | | |
| Example 9 - | 0.62 | 18 | 30.1 | 17.0 | 33 | 70.5 |
| Process B | | | | | | |
| Comparative | 0.64 | 85 | 31.2 | 19.6 | 65 | 68 |
| example 1 | | | | | | |
| Comparative | 0.49 | 23 | 30.6 | 15.3 | 18 | 58 |
| example 2 | | | | | | |
| Comparative | 0.62 | 48 | 29.5 | 18.6 | 52 | 71 |
| example 3 | | | | | | |
| Comparative | 0.60 | 42 | 30.1 | 18.2 | 55 | 68 |
| example 4 | | | | | | |
| Comparative | 0.62 | 45 | 30.1 | 19.0 | 47 | 65 |
| example 5 | | | | | | |
| Comparative | 0.60 | 40 | 30.4 | 18.0 | 36 | 64 |
| example 6 | | | | | | |
| Comparative | 0.60 | 42 | 30.2 | 18.5 | 39 | 65 |
| example 7 | | | | | | |
| Comparative | 0.57 | 37 | 30.0 | 17.4 | 30 | 67 |
| example 8 | | | | | | |

Referring to Table 1, it is confirmed that Examples 1 to 9 exhibit an improved absorbency under load while other physical properties such as the centrifuge retention capacity, liquid permeability, surface tension and bulk density are equal to or higher than those of Comparative Examples 1 to 8, except Comparative Example 2.

In the case of Comparative Example 2, due to excessive use of the surfactant, the bulk density is low and the physical properties such as absorbency under load and liquid permeability are deteriorated.

On the other hand, in view of Comparative Example 5, in which bubbles were generated without a mechanical foaming process showed that despite the use of a large amount of foaming agent and surfactant, absorbency under load for instance was much slower in comparison with Processes A and B in Examples 1-9.

In Comparative Examples 6-7, in which the step of generating microbubbles by ultrasonic wave was not performed, the vortex time was as low as 40 seconds. However, in comparison with Process B in Examples 6-9, the vortex time is 35 seconds or lower demonstrating the vortex time in Process B Examples 6-9 are superior compared with the vortex times of Comparative Examples 6-7.

In Comparative Example 8 where the two-stage bubble generating step was performed without injecting the inorganic fine particles, the absorbency under load was faster than that of Comparative Example 6 or 7. The absorbency under load of Comparative Examples 6-8, however, did not improve to the level of Process A Examples 1-5 and Process B Examples 7-9. In addition, in Comparative Examples 5-8, a large amount of surfactant was used and thus the surface tension was lowered to or below 67 mN/m. The use of a large amount of surfactant in Comparative Examples 5-8 explains a lower surface tension average when compared with Process A Examples 1-5 and Process B Examples 6-9.

### Embodiments

1. An absorbent article comprising: a topsheet; backsheet; and an absorbent core disposed between the topsheet and backsheet wherein the absorbent core comprises:
   a fibrous material and,
   a particulate super absorbent polymer composition comprising;
   a base polymer powder including a first cross-linked polymer of a water-soluble ethylenically unsaturated monomer having an acidic group of superabsorbent polymer composition which has an absorption rate (also known as “vortex time”) measured by a Vortex Time test method of 5 to 35 seconds, a surface tension of 65 to 72 mN/m, a bulk density of 0.50 to 0.65 g/ml, a centrifuge retention capacity (CRC) of 23 g/g or more, a absorbency under load (AUL) at 0.9 psi of 14 g/g or more, a gel bed permeability (GBP) of 10 darcies or more, and a particle size of 150 to 850 μm wherein the particulate superabsorbent polymer composition comprises particles having a particle size of 600 μm or more make up less than 12 wt % of the composition, and particles having a particle size of 300 μm or less make up less than 20 wt % of the composition.
2. The absorbent article according to claim 1, wherein the fibrous material includes absorbent fibers, synthetic polymer fibers, or a combination thereof
3. The absorbent article according to claims 1-2, wherein the particulate super absorbent polymer composition comprising from about 20 wt % to about 90 wt % of the absorbent core.
4. The absorbent article according to claims 1-3, wherein the particulate super absorbent polymer composition comprising a centrifuge retention capacity (CRC) of 25 to 35 g/g.
5. The super absorbent article according to claims 1-4, wherein the particulate super absorbent polymer composition comprising an absorbency under load (AUL) at 0.9 psi of 16 to 23 g/g.
6. The super absorbent article according to claims 1-5, wherein the particulate super absorbent polymer composition comprising a gel bed permeability (GBP) of 25 to 50 Darcy.

What is claimed is:

1. A absorbent article comprising: a topsheet; backsheet; and an absorbent core disposed between the topsheet and backsheet wherein the absorbent core comprises:
a fibrous material and,
a particulate super absorbent polymer composition comprising;
a base polymer powder including a first cross-linked polymer of a water-soluble ethylenically unsaturated monomer having an acidic group of superabsorbent polymer composition which has an absorption rate (also known as “vortex time”) measured by a Vortex Time test method of 5 to 35 seconds, a surface tension of 65 to 72 mN/m, a bulk density of 0.50 to 0.65 g/ml, a centrifuge retention capacity (CRC) of 23 g/g or more, a absorbency under load (AUL) at 0.9 psi of 14 g/g or more, a gel bed permeability (GBP) of 10 darcies or more, and a particle size of 150 to 850 μm wherein the particulate superabsorbent polymer composition comprises particles having a particle size of 600 m or more make up less than 12 wt % of the composition, and particles having a particle size of 300 m or less make up less than 20 wt % of the composition.

2. The absorbent article according to claim 1, wherein the fibrous material includes absorbent fibers, synthetic polymer fibers, or a combination thereof

3. The absorbent article according to claim 1, wherein the particulate super absorbent polymer composition comprising from about 20 wt % to about 90 wt % of the absorbent core.

4. The absorbent article according to claim 1, wherein the particulate super absorbent polymer composition comprising a centrifuge retention capacity (CRC) of 25 to 35 g/g.

5. The absorbent article according to claim 1, wherein the particulate super absorbent polymer composition comprising an absorbency under load (AUL) at 0.9 psi of 16 to 23 g/g.

6. The absorbent article according to claim 1, wherein the particulate super absorbent polymer composition comprising a gel bed permeability (GBP) of 25 to 50 Darcy.
